(19) United States
(12) Patent Application Publication
(10) Pub. No.: US 2014/0357960 A1
(43) Pub. Date: Dec. 4, 2014

(54) Methods and Systems for Synchronizing Repetitive Activity with Biological Factors

(71) Applicants: James William Phillips, Fountain Valley, CA (US); Yi Jin, Irvine, CA (US)

(72) Inventors: James William Phillips, Fountain Valley, CA (US); Yi Jin, Irvine, CA (US)

(21) Appl. No.: 13/907,900

(22) Filed: Jun. 1, 2013

(51) Int. Cl. A61B 5/00; A61B 5/0205; A61B 5/1455; A61B 5/113; A61B 5/0482; A61B 5/0402

(52) U.S. Cl. CPC A61B 5/486; A61B 5/0482; A61B 5/0402; A61B 5/14551; A61B 5/1135; A61B 5/02055; A61B 5/6801; A61B 5/7405; A61B 5/742; A61B 5/7455
USPC 600/301; 600/300; 600/508; 600/529; 600/545; 600/519; 600/528; 600/324; 600/534

(57) ABSTRACT

A method and device is described, which measures and records one or more repetitive biological signals, such as heartbeat, breathing rate, and/or intrinsic brainwave frequency, and uses these tempos and timing information as a feedback mechanism to an individual doing one or more repetitive motion activities, in order to synchronize the activities with the repetitive biological signals, or a simple ratio of harmonics or sub-harmonics thereof. The feedback is achieved through a visual, audio, or tactile signal that indicates to the individual pacing information for precisely when to perform the activity. The purpose of synchronizing repetitive motion activity to biological activity is to optimize the efficiency of the system as a whole, reducing energy consumption and promoting calm and focused performance. Repetitive motion activities include but are not limited to breathing, running, bicycling, swimming, walking, hiking, jump rope, and rowing.

### CROSS REFERENCE TO RELATED APPLICATIONS

This application claims priority to U.S. Provisional Application No. 61/655,021, filed Jun. 4, 2012, the entire disclosure of which is incorporated by reference.

### FIELD OF THE INVENTION

This invention relates to the field of health and fitness and more particularly to methods for improving human performance based on synchronization of human activity with natural biological activity.

### BACKGROUND OF THE INVENTION

It has been recognized that human athletic performance depends in part on the extent to which activity engaged in by the athlete is synchronized with other biological factors, such as heart rate, and that a harmonic relationship exists between different aspects of biological activity. A need exists for methods and systems for establishing and taking advantage of synchronization.

The concept of active pacing to synchronize movement with heartbeat has been the subject of some research. Early in 1921, Coleman observed that a subject who always became breathless when climbing a hill, was able to perform the same task without breathlessness after he started breathing and stepping in unison with his pulse. Coleman also observed that the rise in blood pressure of the subject was only half as great. See Coleman, The Psychological Significance of Bodily Rhythms, Journal of Comparative Psychology 1(3) (1921), 213-220. Others have investigated the natural tendency of the heart to follow stride rate when performing a rhythmic activity. See Kirby et al, Coupling of cardiac and locomotor rhythms, Journal of Applied Physiology 66(1) (1989), 323-329, and Nomura et al, Comparison of cardiolocomotor synchronization during running and cycling, European Journal of Applied Physiology 89(3-4) (2003), 221-229.

In one example, the heart rate is measured and signals to the user when the heart rate is within or outside a target zone (U.S. Pat. No. 8,101,843). Others have created systems with the ability to adjust music tempo based on heart-rate, which uses a more up-beat tempo of music to encourage the user to increase activity until the target hart rate is achieved, and then switch to a lower tempo music (Pat #20070074619, #20070044641, #20030066413), or to match the music to the pace of exercise, ie. footfall (U.S. Pat. No. 7,973,231). Others have created systems that select a particular type of music depending on the difference between a heart rate and a target zone (Pat #20060169125, U.S. Pat. No. 6,572,511). One system has been devised which matches music tempo to the heart rate in order to improve mood and decrease depression, locating the music beat midway between heart beats (U.S. Pat. No. 7,207,935). Another system matches music to the tempo of the heart rate (or a desired heart rate) to calm the person and allow them to operate according to their own “internal clock” (U.S. Pat. No. 6,230,047). Another system matches music to the heart rate, then gradually increases or decreases tempo in an attempt to alter the heart rate (U.S. Pat. No. 5,267,942). One can use other data to adjust the tempo of music or rhythmic beats. For example, one system was designed to create a beat in sync with body movements, like the arms or legs, to attain optimal rhythmic movement (U.S. Pat. No. 4,776,323).

### SUMMARY OF THE INVENTION

Methods and systems are provided herein for providing feedback to a user, such as an athlete, such that the user consciously alters physical activity, e.g., footfalls in running or strokes in swimming or rowing. Providing this feedback directly to the user allows for efficient synchronization of all elements of the body, the voluntary elements (footfalls, arm movements, breathing) with the autonomic (heart rate, intrinsic brainwave frequency).

Methods and systems are provided herein whereby one or more rhythmic biological signals are recorded and a rhythmic stimulus is generated that is presented to the user as feedback to allow the user to perform a repetitive motion activity such that the frequency of the repetitive motion matches or is close to a common fraction multiplied by the biological signal frequency.

In broad terms, the present invention comprises a system and method to measure and record one or more repetitive biological signals, and to use these signals as a feedback mechanism that allows the user to perform one or more repetitive motion activities in sync with the biological rhythm, or a simple ratio of harmonics or sub-harmonics thereof. The purpose of this is to allow the body to perform in a state in which all elements are in resonant harmony, thereby lowering the overall energy consumption for the user and allowing for improved performance in the repetitive motion task, as well as to improve wellness, focus, and concentration, and to help reduce injuries. Advantages of performing repetitive motion in sync with the biological rhythm include, but are not limited to, a reduction of stress on the body as a result of the activity, improved efficiency of the activity, increased blood perfusion to the muscles, lowered oxygen uptake, and reduction of blood pressure variability.

When a user performs a repetitive activity, the heart rate is not always completely rhythmic. There can be beats that occur earlier or later than would be predicted, but the average heart rate remains relatively consistent. These early or late beats may be caused by performing the repetitive motion activity out of sync with the heartbeat. Therefore, one benefit of matching the repetitive motion to be in sync with the heart beat is to reduce or eliminate the number of early or late beats, thus making the system more efficient, reducing heart rate variability, and improving the performance of the user.

Performing a repetitive motion activity in synchrony with one or more biological signals can cause the brainwaves to become more synchronous and rhythmic, due to their harmonic relationship with other biological signals. Improving the synchronicity and coherence of the brain can help to reduce the symptoms of mental disorders, including, but not limited to, depression, anxiety, obsessive-compulsive, seizure, Parkinson's disease, ADHD, autism, substance abuse, head injury, Alzheimer's disease, eating disorder, sleep disorder, tinnitus, or any combination thereof.

The activity of a user focusing on a repetitive motion activity is similar in process to those practicing meditation. In meditation, an individual concentrates on a specific thing to the exclusion of all others. Some examples include chanting a mantra, listening to a repetitive sound, and rocking back and forth. In this invention, since the user focuses on the timing of a repetitive motion activity, it is possible for the individual to gain the benefits of a meditative state while performing the activity. Therefore, the user may develop a sense of calm and well being, and not be as acutely aware of any discomfort that may be felt as a result of performing the activity itself. One non-limiting example is a runner who focuses on the timing of his or her footfalls. By focusing on this, the runner achieves a meditative state and is not as acutely aware of being fatigued or having muscle soreness. Benefits of achieving a meditative state include, but are not limited to, promoting relaxation, stress relief, improved focus and concentration, wellness, and a reduction of awareness of discomfort, fatigue, and muscle soreness.

The frequency of the stimulus feedback to the user may be equal to a simple integer ratio of harmonics and sub-harmonics of the biological signal. In one aspect, the frequency could be equal to the frequency of the biological signal multiplied by the common fraction with the smallest denominator that falls within a pre-set comfortable range for the user. The physiological signal may be, for example, heart rate, breathing rate, or the intrinsic EEG frequency within a specified EEG band.

The feedback given to the user can be an audio or visual stimulus, or a tactile stimulus (e.g., vibration, tapping), which has a noticeable beat, allowing the user to time the rhythmic activity to match the beat. The beat may be either an independent item, such as a flash of light or clicking sound, or it may be contained within other content, such as music that is modulated such that the natural musical beat matches the desired stimulation, or within a video, which provides a flashing symbol on the screen along with the video stream that the user can watch to provide timing for the rhythmic activity.

In one aspect, audio stimulus may be used for pacing the activity. Some examples include, but are not limited to, drum beats, tones, or taps. The stimulus may be provided by modulating music, where the tempo of the music is shifted to be equal to, or a harmonic or sub-harmonic of the desired stimulus frequency. The stimulus may also be provided by selecting music that has a tempo that matches, or is close to, the pacing stimulus, or a harmonic or sub-harmonic thereof. The music is chosen from a set with a variety of tempos. It is also possible to use a combination of music selection and music modulation, where a song is selected with a tempo that falls within a range around the desired stimulus frequency, or a harmonic or sub-harmonic thereof, and then modulating the song so that the tempo matches, or is close to, the desired stimulus frequency, or a harmonic or sub-harmonic thereof. In the previous two examples using song selection, a new song could be selected when the previous song ends, or when the stimulus frequency exceeds a specified range from the natural tempo of the previous song. A beat sound can also be performed by introducing a variability or warble onto the music itself.

In one aspect, pacing stimulus may be delivered mechanically. The stimulus may be given by pulsing a diaphragm close to or on the skin to give a tapping feeling to the subject. The tapping location includes, but is not limited to, the wrist, the arm, the head, the ear, and the torso. The stimulus may also be given using vibration of a mechanism placed close to or on the skin of the user.

In one aspect, pacing stimulus may be delivered visually. Some examples include, but are not limited to, flashing a light, flashing a LCD pixel, flashing an icon on a screen, and moving an object at the desired pacing stimulus, or a harmonic or sub-harmonic thereof. An object on a video screen may be moved or flashed at the desired pacing stimulus, either on its own, as part of information presented to the user, part of a game or activity, or part of an entertaining video.

The one or more repetitive motion activities that the user adjusts to match the stimulus signals can be any for which the user is able to adjust the timing. One non-limiting example is breathing, where the user breathes in and out in precise timing with the provided stimulus. Several other non-limiting examples exist in sporting activities, including running (e.g., footfalls, arm swings), bicycling (e.g., pedal revolutions), swimming (e.g., arm strokes, leg kicks, breathing intake), walking or marching (e.g., footfalls, arm swings), hiking (e.g., footfalls, arm swings), jumping rope (e.g., jumps), aerobics (leg movement, arm movement), dancing (e.g., body motion), boxing (e.g., bag strikes), and rowing (e.g., oar strokes). Other non-limiting example activities include hammering (e.g., hammer strokes), and typing (e.g., keystrokes).

It is possible to provide a stimulus for more than one repetitive motion activity, by using a combination of two different stimulation techniques. For example, footfalls and breathing may be controlled by the user based on stimulation feedback that is based on a measure of the heartbeat during running. This can be done with two overlapping audio beats or tones, one to indicate when each footfall is to occur and the other to indicate when a breath should be taken, or using two distinct beat sounds, one indicating that the user should breathe in and one that the user should breathe out. It could also be done with two video icons, one that flashes in time to each footfall, and one that flashes in time to each breath. It could also be done with a combination of video and audio stimulation, such as having an audio beat for footfalls and a video icon that flashes, moves, or changes shape with each breath.

Many ways exist to measure biological signals that can then be used to create the feedback signal. For heart rate, the device could use a set of electrocardiogram (ECG) electrodes close to or touching the skin in an area that provides an electric ECG signal. Some non-limiting examples include a torso strap monitor with electrodes touching the chest. In addition electrodes could be incorporated into a wristband, armband, neckband, or clipped to the earlobe.

In one non-limiting aspect, the heart rate could be detected using a pulse oximeter close to or touching the skin in a location where a signal may be obtained. The pulse oximeter may be clipped to the ear, inserted in the ear, clipped or pressed to the finger, clipped to the toe, or incorporated as part of a wristband or wristwatch, touching the skin of the wrist.

In one non-limiting aspect, the heart rate could be detected using sound generated by the beating heart. For example, a sensing diaphragm, similar to a sphygmomanometer or microphone, could be incorporated into a torso strap or a wristband or wristwatch.

In one non-limiting aspect, breathing can be detected using electromyography (EMG), torso girth sensor, microphone, or oxygen sensor, or a combination thereof.

In one non-limiting aspect, stride rate can be recorded using a pedometer, which communicates wirelessly, wired, or is incorporated into the device. For example, the pedometer may be worn in or on the shoe, on the wrist, clipped to the belt, or carried.

In one non-limiting aspect, the brainwaves may be recorded using an electroencephalogram with bio-potential electrodes attached to the scalp, using capacitive electrodes at or near the scalp, or using a magneto-encephalogram (MEG).

In one non-limiting aspect, location during the activity may be recorded using a global positioning system (GPS), cell-tower triangulation, or an accelerometer that detects body motion.

In one non-limiting aspect, speed may be recorded using a change in location over time, or with an accelerometer that detects body motion.

Other measurements may also be included with the device, including but not limited to a measurement of atmospheric temperature, humidity, and skin temperature.

The device can be worn entirely by the user. Non-limiting examples include a wristband, such as a wristwatch, activity tracker, heart rate monitor, GPS, or pedometer. The device could be incorporated into headphones, such as radio headphones, noise cancellation headphones, or MP3 player headphones. Also, it could be a tabletop unit, or it could be incorporated into an exercise machine, such as a treadmill, elliptical machine, stair stepper, bicycle, or rowing machine. It could also be incorporated into an application on a cellular phone or other mobile device, such as a PDA, tablet PC, or MP3 player. In addition, it could be incorporated into an existing entertainment system, such as a television, video monitor, or stereo system. Thus, in various non-limiting embodiments, interfaces (including hardware, networking interface, and/or software interfaces) may be provided between a system or component for determining a biological condition, such as heart rate, a system or component for determining one or more stimulus signals, and one or more systems or components of any of the types of devices described herein. Such interfaces may include, without limitation, wireless and wired communication interfaces.

In one aspect, the device incorporates a heart monitor, either internally or through a wired or wireless connection. One non-limiting example includes a device incorporated into headphones with a pulse oximeter clipped to the earlobe or inserted in the ear that is connected by a wire to the device. Another non-limiting example is a device with clip-on or adhesive electrodes that detect heartbeat using electrical activity sensed on the skin, such as a torso heart monitor, wristband, or armband. Another non-limiting example is a device that communicates wired or wirelessly with a heart monitor, such as with a torso strap heart monitor, ECG electrodes, or pulse oximeter.

In one aspect, the device comprises a pulse-oximeter clipped onto the ear, which sends heartbeat signals to a central processor, which uses an internal algorithm to generate the stimulus signal at the heart rate or a simple common fraction of harmonics or sub-harmonics thereof. The stimulus signal is an electronic beat, which is played through headphones for the user. Breathing stimulus may also be added to the beat signal, such as with alternating tones to indicate breath inhalation or exhalation. The electronics can be incorporated into the headphones or carried separately.

The biological signals can be sent wired or wirelessly from the biological sensor to the processor. In one aspect, a pulse oximeter attached to the ear transmits a signal wirelessly to a portable cellular phone or MP3 player that receives the signal and uses an application to generate the stimulation signal that is then played through the headphones worn by the person.

The system can be incorporated into exercise equipment. In one aspect, an ECG sensor transmits wirelessly to a receiver incorporated into the exercise equipment. The processor in the module uses its internal algorithm to generate the stimulus signal or signals, which can then be sent to the user. Some non-limiting ways of doing this are wirelessly transmitting to headphones worn by the user, transmitting to the user's headphones through a wired connection, playing the sound through speakers incorporated into the exercise equipment, or using a visual signal to the user via a LED or video display, or any combination thereof.

It is possible to measure the repetitive motion activity of the user. Some ways of accomplishing this are with a motion sensor placed on the body part that undergoes the motion, or by using an EMG sensor to detect muscle contractions, with RF motion sensors, or with a camera and video processing software to detect motion. It is possible to give the user additional feedback as to how well they are matching the stimulus signal with their activity. In one aspect of the device, an accuracy measure can be incorporated into the processor that adjusts the tone of the audio beat signal such that the tone changes depending on the accuracy of the user to match the stimulus signal. In another aspect of the device, an accuracy measure can be incorporated into a visual cue, which turns a different color depending on the accuracy of the user in matching the stimulus signal.

In one aspect, the device announces to the user at regular intervals, or as requested, the statistics regarding their performance. These statistics may include at least one of average heart rate, peak heart rate, minimum heart rate, distance covered, average speed, average pace frequency, accuracy of pace to the specified pace, and motivational slogans.

In one aspect, the device incorporates a means by which change in altitude is measured, thereby allowing the device to determine when the user is going uphill or downhill or is moving on flat ground. The device incorporates an option to change at least one of the normal pace frequency, the minimum acceptable pace frequency, and the maximum acceptable pace frequency, depending on the slope of the incline or decline.

In one aspect, in order to determine the stimulation frequency based on the measured biological beat frequency, the algorithm may take into account the following: Biological Signal Beat Frequency (BF); Normal Pace Frequency (NP); Maximum Acceptable Pace Frequency (PH); and/or Minimum Acceptable Pace Frequency (PL).

The NP depends on the type of repetitive motion activity and the preference of the user, and possibly the environment (slope, temperature, etc.). The NP is the average pace frequency that the user would expect to maintain without feedback stimulation. For example, if the user is running, the NP may be approximately 180 spm, so NP=180 in this case. The user may not feel comfortable running with a pace faster than 200 spm or slower than 150 spm. Therefore, PH=200 and PL=150.

The algorithm attempts to find the simplest common fraction (smallest denominator) of harmonics and sub-harmonics of BF that fall within the acceptable range around NP. If multiple fractions with the same denominator fall within the range, the stimulation frequency (SF) may optionally be set to the one that is closest to NP within this range. Note that the ratio will not change until the calculated SP is out of range. This prevents the stimulation frequency varying too frequently.

The acceptable range can be set by the user, either by using trial and error to determine the parameters that the user finds acceptable or based on other factors, such as settings that work for other similar users. Alternately, the user could use feedback from the device to assist in setting parameters. Some non-limiting examples include a metric showing how well the user matches one or more of his or her repetitive motion to the pacing signal, such as stride rate and breathing rate.

In one aspect, the parameters of the algorithm, such as the acceptable range for pacing frequency, can be adjusted automatically by the device. For example, the parameters could change based on atmospheric temperature, altitude, slope, atmospheric humidity, the user's location or distance traveled, the user's speed, or time of the activity, or any combination thereof. In another non-limiting example, the parameters of the algorithm can be adjusted based on the physical condition of the user, such as body temperature, heart rate, accuracy of matching the pacing stimulus such as stride rate or breathing, breathing volume, or brainwave activity as measured by EEG.

In one aspect, the phase of the pacing stimulus is shifted so that the repetitive motion activity is brought into a specified phase relationship with the biological signal. For example, the stimulus could be adjusted so that a runner's strides are in phase with the stimulus signal, and therefore are in phase with the runner's heartbeat, thereby optimizing blood flow to the muscles.

### BRIEF DESCRIPTION OF DRAWINGS

FIG. 1 is a flowchart showing one aspect of the device in which the processing of a biological signal occurs to create a stimulus that is played through a speaker as feedback for the user.

FIG. 2 shows a flowchart for the processor (105) from FIG. 1 in which a biological signal is processed.

FIG. 3 shows a possible flowchart for an algorithm to create the stimulation frequency.

FIG. 4 shows one aspect, where an ear-clip pulse oximeter records the heart rate, and electronics and processor are embedded in or on one of the earphones.

FIG. 5 shows an alternate aspect, in which a pulse oximeter is inserted into the ear with earphones.

FIG. 6 shows an alternate aspect, in which a torso-mounted ECG sensor transmits the beat signal to the portable device such as a cellular phone.

FIG. 7 shows an alternate aspect, in which the user is on a treadmill.

FIG. 8 shows an alternate aspect, in which the device is contained in a wristband.

FIG. 9 shows an alternate aspect, in which the device is incorporated into a smart phone application.

FIG. 10 shows an alternate aspect, in which the device is incorporated into a smart phone application.

FIG. 11 shows an alternate aspect, in which the device is incorporated into a smart phone application.

FIG. 12 shows a representative example of a subject's heart rate during sham versus active pacing.

### DETAILED DESCRIPTION

While certain embodiments have been provided and described herein, it will be readily apparent to those skilled in the art that such embodiments are provided by way of example only. It should be understood that various alternatives to the embodiments described herein may be employed, and are part of the invention described herein.

The methods and systems described herein take advantage of the phenomenon of resonance. A harmonic relationship exists between different functional regions of the body. The intrinsic frequency of the brain, ie., the alpha frequency, is approximately the 8th harmonic of the heart rate. The heart rate is approximately the 5th harmonic of the breathing rate, and the breathing rate is approximately the 5^{th} harmonic of the intestinal movement frequency. In general, a system operates more efficiently when all elements of the system work synchronously. An efficient system uses less energy and allows for improved performance of each element individually.

Resonant systems are abundant in nature, with autonomous elements operating in concert to reduce the energy consumption of the system as a whole. For an individual performing a repetitive motion activity, the ability to perform that activity synchronously to natural biological activity will help to improve the efficiency of the activity and reduce the stress on other elements of the body. This may result in improved performance of the activity itself and promote wellness, focus, and concentration, and help to reduce injuries.

In one example, a runner maintains a heart rate in the zone from 100-140 beats per minute (bpm). Assume 120 bpm in this example. The average running rate is approximately 180 steps per minute (spm), 90 left foot, and 90 right foot. Therefore, the optimal running rate to maintain synchrony between the heart and the footfalls is 3 running steps for every 2 beats. If the runner's heart rate increased to 130 bpm, then the optimal running rate is no longer 180 spm. It has changed to 195 spm to maintain 3 running steps for every 2 beats. Alternatively, the optimal rate could decrease to 130 spm, or 1 running step per beat. Maintaining this relationship between heart rate and running spm allows the system to perform as a synchronous whole, with all elements contributing to the central rhythm. Since a synchronous system is more efficient than an asynchronous one, the runner uses less energy to maintain the same speed, which allows for improved performance of the individual, and may help to prevent injuries, and reduce stress on body organs, such as the heart.

When running, the body undergoes significant impact, due to the natural bouncing during the activity. It has been found that blood pressure changes dynamically due to change in direction and change in position. It is also well known that blood pressure changes during each heartbeat. Therefore, timing the repetitive activity (e.g, footfall during running) to coincide with the user's heartbeat can allow the heart to contract at the optimal time in order to optimize variation in blood pressure, thereby lowering the stress on the heart and the rest of the body.

If we include a second repetitive motion activity along with the running spm, this activity would be more efficient if it is performed synchronously as well. For example, breathing rate has been shown to be optimal at one breath for every 5 heartbeats. If the runner whose heart rate is 120 bpm breathes 24 times per minute, these breaths should occur synchronous to the heart beat. The rhythmic breathing adds to the synchronous system to improve efficiency further and reduce stress on the system as a whole.

Heart rate monitoring is widely available to the average consumer for decades, and those of ordinary skill in the art use heart rate monitoring to manage fitness activities.

In embodiments, methods and systems described herein may match stride rate or other voluntary muscular movements on coupling at a 1:1 ration or at a different ratio, allowing athletes to match stride to a simple ratio of a biological signal, for example, 12, 2:3, or 3:2.

Provided herein is a method whereby one or more rhythmic biological signals are recorded, and a stimulation signal is generated that is presented to the user as feedback to allow the user to perform a repetitive motion activity in sync with the biological signal, such that the frequency of the repetitive motion matches a common fraction, or integer ratio, multiplied by the biological signal frequency.

In broad terms, the present invention comprises a system and method to measure and record one or more repetitive biological signals, and to use these signals as a feedback mechanism that allows the user to perform one or more repetitive motion activities in sync with the biological rhythm, or a simple ratio of harmonics or sub-harmonics thereof. The purpose of this is to allow the body to perform in a state in which all elements are in resonant harmony, thereby lowering the overall energy consumption for the user and allowing for improved performance in the repetitive motion task, as well as to improve wellness, focus, and concentration, and to help reduce injuries. Advantages of performing repetitive motion in sync with the biological rhythm include, but are not limited to, a reduction of stress on the body as a result of the activity, improved efficiency of the activity, improved performance of the activity, increased blood perfusion to the muscles, lowered oxygen uptake, and reduction of blood pressure variability.

Provided herein is a method through the use of the feedback stimulation causes at least one biological signal to become more regular and have fewer spurious or arrhythmic beats. When a user performs a repetitive activity, the heart rate is not always completely rhythmic. There can be beats that occur earlier or later than would be predicted, but the average heart rate remains relatively consistent. These early or late beats may be caused by performing the repetitive motion activity asynchronous to the heart beat. Therefore, one benefit of matching the repetitive motion to be in sync with the heart beat is to reduce or eliminate the number of early or late beats, thus making the system more efficient, reducing heart rate variability, and improving the performance of the user.

Performing a repetitive motion activity in synchrony with one or more biological signals can cause the brainwaves to become more synchronous and rhythmic, due to their harmonic relationship with other biological signals. Improving the synchronicity and coherence of the brain can help to reduce the symptoms of mental disorders, including, but not limited to, depression, anxiety, obsessive-compulsive, seizure, Parkinson's disease, ADHD, autism, substance abuse, head injury, Alzheimer's disease, eating disorder, sleep disorder, tinnitus, or any combination thereof.

The activity of a user focusing on a repetitive motion activity is similar in process to those practicing meditation. In meditation, an individual concentrates on a specific thing to the exclusion of all others. Some examples include chanting a mantra, listening to a repetitive sound, and rocking back and forth. In this invention, since the user focuses on the timing of a repetitive motion activity, it is possible for the individual to gain the benefits of a meditative state while performing the activity. Therefore, the user may develop a sense of calm and well-being, and not be as acutely aware of any discomfort that may be felt as a result of performing the activity itself. One non-limiting example is a runner who focuses on the timing of his or her footfalls. By focusing on this, the runner achieves a meditative state and is not as acutely aware of being fatigued or having muscle soreness. Benefits of achieving a meditative state include, but are not limited to, promoting relaxation, stress relief, improved focus and concentration, wellness, and a reduction of awareness of discomfort, fatigue, and muscle soreness.

The stimulus provided to the user specifies exactly when one or more repetitive motions are to be performed. The frequency of the stimulus feedback to the user may be equal to a simple integer ratio of harmonics and sub-harmonics of the biological signal. In one aspect, the frequency could be equal to the frequency of the biological signal multiplied by the common integer fraction with the smallest denominator that falls within a pre-set comfortable range for the user. The physiological signal may be, for example, heart rate, breathing rate, or the intrinsic EEG frequency within a specified EEG band.

In the case that the stimulation is only used as a means of enhancing meditation, it does not need to be based on a biological signal. Instead, the stimulation could be set to the normal pace for the repetitive motion activity (ie., the pace that would occur if no stimulation were present). In this case, a device that provides the stimulation would not require a means of recording the biological signal.

The feedback given to the user can be at least one of an audio or visual stimulus, or a tactile stimulus (e.g., vibration, tapping), which has a noticeable beat, allowing the user to time the rhythmic activity to match the beat. The beat may be either an independent item, such as a flash of light or clicking sound, or it may be contained within other content, such as music that is modulated such that the natural musical beat matches or is close to the desired stimulation, or within a video, which provides a flashing symbol on the screen along with the video stream that the user can watch to provide timing for the rhythmic activity.

In one aspect, audio stimulus may be used for pacing the activity. Some examples include, but are not limited to beeps, tones, drumbeats, thumps, hisses, and voices. The stimulus may be provided by modulating music, where the tempo of the music is shifted to be equal or close to, or a harmonic or sub-harmonic of the desired stimulus frequency. The stimulus may also be provided by selecting music that has a tempo that matches, or is close to, the pacing stimulus. The music is chosen from a set with a variety of tempos. It is also possible to use a combination of music selection and music modulation, where a song is selected with a tempo that falls within a range around the desired stimulus frequency, or a harmonic or sub-harmonic thereof and then the song is modulated so that the tempo matches, or is close to, the desired stimulus frequency, or a harmonic or sub-harmonic thereof. In the previous two examples using song selection, a new song could be selected when the previous song ends, or when the stimulus frequency exceeds a specified range from the natural tempo of the previous song. A beat sound can also be performed by introducing a variability or warble onto the music itself.

In one aspect, pacing stimulus may be delivered mechanically. The stimulus may be given by pulsing a diaphragm close to or on the skin to give a tapping feeling to the subject. The tapping location includes, but is not limited to, the wrist, the arm, the head, the ear, and the torso. The stimulus may also be given using vibration of a mechanism placed close to or on the skin of the user.

In one aspect, pacing stimulus may be delivered visually. Some examples include, but are not limited to, flashing a light, flashing a LCD pixel, flashing an icon on a screen, and moving an object at the desired pacing stimulus, or a harmonic or sub-harmonic thereof. An object on a video screen may be moved or flashed at the desired pacing stimulus, either on its own, as part of information presented to the user, part of a game or activity, or part of an entertaining video.

The one or more repetitive motion activities that the user adjusts to match the stimulus signals can be any for which the user is able to adjust the timing. One non-limiting example is breathing, where the user breathes in and out in precise timing with the provided stimulus. Several other non-limiting examples exist in sporting activities, including running (e.g., footfalls, arm swings), bicycling (e.g., pedal revolutions), swimming (e.g., arm strokes, leg kicks, breathing intake), walking or marching (e.g., footfalls, arm swings), hiking (e.g., footfalls, arm swings), jumping rope (e.g., jumps), aerobics (leg movement, arm movement), dancing (e.g., body motion), boxing (e.g., bag strikes), and rowing (e.g., oar strokes). Other non-limiting example activities include hammering (e.g., hammer strokes), and typing (e.g., keystrokes).

It is possible to provide a stimulus for more than one repetitive motion activity, by using a combination of two different stimulation techniques. For example, footfalls and breathing may both be controlled by the user based on stimulation feedback that is based on a measure of the heartbeat during running. This can be done with two overlapping audio beats or tones, one to indicate when each footfall is to occur and the other to indicate when a breath should be taken, or using two distinct beat sounds, one indicating that the user should breathe in and one that the user should breathe out. It could also be done with two video icons, one that flashes in time to each footfall, and one that flashes in time to each breath. It could also be done with a combination of video and audio stimulation, such as having an audio beat for footfalls and a video icon that flashes, moves, or changes shape with each breath.

Many ways exist to measure biological signals that can then be used to create the feedback signal. Heart rate could be recorded using at least one of electrocardiogram (ECG) electrodes, pulse oximetry, and a microphone. The device could use a set of electrocardiogram (ECG) electrodes close to or touching the skin in an area that provides an electric ECG signal. Some non-limiting examples include a torso strap monitor with electrodes touching the chest. In addition electrodes could be incorporated into a wristband, armband, neckband, or clipped to the earlobe.

In one non-limiting aspect, the heart rate could be detected using a pulse oximeter close to or touching the skin in a location where a signal may be obtained. The pulse oximeter may be clipped to the ear, inserted in the ear, clipped or pressed to the finger, clipped to the toe, or incorporated as part of a wristband or wristwatch, touching the skin of the wrist.

In one non-limiting aspect, the heart rate could be detected using sound generated by the beating heart. For example, a sensing diaphragm, similar to a sphygmomanometer, or microphone, could be incorporated into a torso strap or a wristband or wristwatch.

In one non-limiting aspect, breathing can be detected recorded using at least one of electromyography, torso girth sensor, microphone, and oxygen sensor using electromyography (EMG), torso girth sensor, microphone, or oxygen sensor, or a combination thereof.

In one non-limiting aspect, stride rate can be recorded using a pedometer, which communicates wirelessly, wired, or is incorporated into the device. For example, the pedometer may be worn in or on the shoe, on the wrist, clipped to the belt, or carried.

In one non-limiting aspect, the brainwaves may be recorded using an electroencephalogram with bio-potential electrodes attached to the scalp, using capacitive electrodes at or near the scalp, or using a magneto-encephalogram (MEG).

In one non-limiting aspect, location during the activity may be recorded using a global positioning system (GPS), cell-tower triangulation, or an accelerometer that detects body motion.

In one non-limiting aspect, speed may be recorded using a change in location over time, or with an accelerometer that detects body motion.

Other measurements may also be included with the device, including but not limited to a measurement of atmospheric temperature, humidity, wind, and body temperature. This information may be used optionally to adjust at least one of normal pace frequency, maximum acceptable pace frequency, and minimum acceptable pace frequency. Adjustment may be made automatically under the control of a processor, or by user intervention, such as by a user interface button, dial, slide mechanism, touch screen, voice command, or the like.

The device can be worn entirely by the user. Non-limiting examples include a wristband, such as a wristwatch, activity tracker, heart rate monitor, GPS, or pedometer. The device could be incorporated into headphones, such as radio headphones, noise cancellation headphones, or MP3 player headphones. Also, it could be a table-top unit, or it could be incorporated into an exercise machine, such as a treadmill, elliptical machine, stair stepper, bicycle, or rowing machine. It could also be incorporated into an application on a cellular phone or other mobile device, such as a PDA, tablet PC, or MP3 player. In addition, it could be incorporated into an existing entertainment system, such as a television, video monitor, or stereo system.

In one aspect, the device incorporates a heart monitor, either internally or through a wired or wireless connection. One non-limiting example includes a device incorporated into headphones with a pulse oximeter clipped to the earlobe or inserted in the ear that is connected by a wire to the device. Another non-limiting example is a device with clip-on or adhesive electrodes that detect heartbeat using electrical activity sensed on the skin, such as a torso heart monitor, wristband, or armband. Another non-limiting example is a device that communicates wired or wirelessly with a heart monitor, such as with a torso strap heart monitor, ECG electrodes, or pulse oximeter.

In one aspect, the device comprises a pulse-oximeter clipped onto the ear, which sends heart-beat signals to a central processor, which uses an internal algorithm to generate the stimulus signal at the heart-rate or a simple common fraction of harmonics or sub-harmonics thereof. The stimulus signal is an electronic beat, which is played through headphones for the user. Breathing stimulus may also be added to the beat signal, such as with alternating tones to indicate breath inhalation or exhalation. The electronics can be incorporated into the headphones or carried separately.

The biological signals can be sent wired or wirelessly from the biological marker to the processor. In one aspect, a pulse oximeter attached to the ear transmits a signal wirelessly to a portable cellular phone or MP3 player that receives the signal and uses an application to generate the stimulation signal that is then played through the headphones worn by the person.

The system can be incorporated into exercise equipment. In one aspect, an ECG sensor transmits wirelessly to a receiver incorporated into the exercise equipment. The processor in the module uses its internal algorithm to generate the stimulus signal or signals, which can then be sent to the user. Some non-limiting ways of doing this are wirelessly transmitting to headphones worn by the user, transmitting to the user's headphones through a wired connection, playing the sound through speakers incorporated into the exercise equipment, or using a visual signal to the user via a LED or video display.

The system can be worn by a member of the armed forces or other individual who takes part in an endurance hike. By providing audio feedback as cadence to the user, the energy consumption is reduces, the user may go at least one of faster and longer distance without getting as tired.

It is possible to measure the repetitive motion activity of the user. Some ways of accomplishing this are with a motion sensor placed on the body part that undergoes the motion, or by using an EMG sensor to detect muscle contractions, with RF motion sensors, or with a camera and video processing software to detect motion. It is possible to give the user additional feedback as to how well they are matching the stimulus signal with their activity. In one aspect of the device, an accuracy measure can be incorporated into the processor that adjusts the tone of the audio beat signal such that the tone changes depending on the accuracy of the user to match the stimulus signal. In another aspect of the device, an accuracy measure can be incorporated into a visual cue, which turns a different color depending on the accuracy of the user to match the stimulus signal.

In one aspect, the device presents or announces to the user at regular intervals, or as requested, the statistics regarding their performance. These statistics may include at least one of average heart rate, peak heart rate, minimum heart rate, instantaneous heart rate, distance covered, calories burned, average distance per pace, average speed, average pace frequency, stimulus frequency, accuracy of pace to the specified pace, and motivational messages.

In one aspect, the device incorporates a means by which change in altitude is measured, thereby allowing the device to determine when the user is going uphill or downhill or is moving on flat ground. The device incorporates an option to change at least one of the normal pace frequency, the minimum acceptable pace frequency, and the maximum acceptable pace frequency, depending on the slope of the incline or decline.

In order to determine the stimulation frequency based on the measured biological beat frequency, the algorithm may take into account at least one of the following: Biological Signal Beat Frequency (BF); Normal Pace Frequency (NP); Maximum Acceptable Pace Frequency (PH); and/or Minimum Acceptable Pace Frequency (PL).

The NP depends on the type of repetitive motion activity and the preference of the user, and possibly the environment (slope, temperature, etc.). The NP is the average pace frequency that the user would expect to maintain without feedback stimulation. For example, if the user is running, the NP may be approximately 180 spm, so NP=180 in this case. The user may not feel comfortable running with a pace faster than 200 spm or slower than 150 spm. Therefore, PH=200 and PL=150.

The algorithm attempts to find the simplest common integer fraction (smallest denominator) of harmonics and sub-harmonics of BF that fall within the acceptable range around NP. If multiple fractions with the same denominator fall within the range, the stimulation frequency (SF) is set to the one that is closest to NP within this range. Note that the ratio will not change until the calculated SP is out of range. This prevents the stimulation frequency varying too frequently.

The acceptable range can be set by the user, either by using trial and error to determine the parameters that the user finds acceptable or based on other factors, such as what works for similar users, or what is recommended for users of a certain type. Alternately, the user could use feedback from the device to assist in setting parameters. Some non-limiting examples include a metric showing how well the user matches one or more of his or her repetitive motion to the pacing signal, such as stride rate and breathing rate.

In one aspect, the parameters of the algorithm, such as the acceptable range for pacing frequency, can be adjusted automatically by the device. For example, the parameters could change based on atmospheric temperature, altitude, slope, atmospheric humidity, the user's location or distance traveled, the user's speed, or time of the activity, or any combination thereof. In another non-limiting example, the parameters of the algorithm can be adjusted based on the physical condition of the user, such as body temperature, heart rate, accuracy of matching the pacing stimulus such as stride rate or breathing, breathing volume, or brainwave activity as measured by EEG.

In one aspect, the phase of the pacing stimulus is shifted so that the repetitive motion activity is brought into a specified phase relationship with the biological signal. For example, the stimulus could be adjusted so that a runner's strides are in phase with the stimulus signal, and therefore are in phase with the runner's heartbeat, thereby optimizing blood flow to the muscles.

In one aspect, the parameters of a stationary exercise machine can be automatically adjusted based on information gathered from the user by the device. For example, the speed of a treadmill could be adjusted based on a metric related to the ability of the user to match the pacing and/or breathing stimulus signal. In another non-limiting example, the resistance of a stair stepper or elliptical machine could be adjusted so that the user's stride is in phase with their heartbeat.

In one aspect, a bicycle could automatically switch gears based on information gathered on the device. For example, the gears could be switched based on the ability of the user to match the pacing or breathing stimulus. In another non-limiting example, the gears could be switched in order to maintain a desired speed while keeping the pedal rate within the acceptable range defined by the user.

It is also possible to provide group synchronization using this device. Examples include, but are not limited to marching, dancing, and aerobics. In one aspect, each member of the group wears a heart monitor that communicates either directly or indirectly with a central device. The device determines the average or median heart rate of the group and finds the pacing stimulus based on the average. The pacing stimulus is provided to each member of the group, either through a central source, such as a speaker or light, or transmitted back to each member's device so that the device can provide the pacing stimulus to each user. In this way, all members of the group are in sync with the desired pacing frequency.

In one aspect, an application is installed on a portable device, such as a smart phone, MP3 player, or PDA. The application is offered through download from an on-line store. The application could have a pricing structure where a limited version is offered for free, with an upgrade for a nominal fee. For example, the free version could remove support for a heart monitor, and instead require the user to enter a target heart rate at which they expect to perform the activity.

In one aspect the device, an application is installed on a portable device, such as a smart phone, MP3 player, or PDA, that offers downloadable music or streaming music, in which the tempo of the songs match or are close to the desired stimulation frequency. The application could optionally incorporate modulation, shifting the tempo of the song within a range, but selecting a new song at or near the most recent desired stimulation frequency when the previous song ends or when the stimulus frequency exceeds a specified range from the natural tempo of the song.

In one aspect of the device, an application is installed on a portable device, such as a smart phone, MP3 player, or PDA, that chooses songs from a playlist stored on the device in which the tempo of the song matches or is close to the desired stimulation frequency. The application could optionally incorporate modulation, shifting the tempo of the song within a range, but selecting a new song at or near the most recent desired stimulation frequency when the previous song ends or when the stimulus frequency exceeds a specified range from the natural tempo of the song.

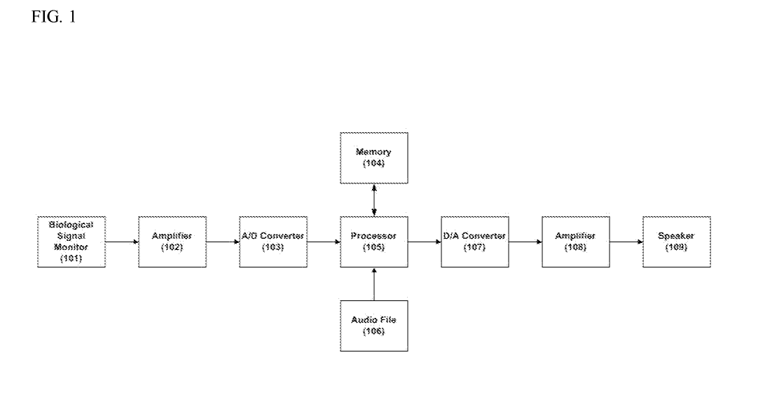

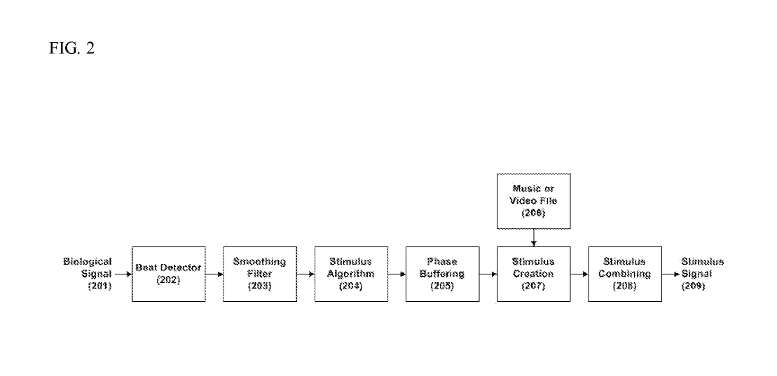

Reference is now made to FIG. 1, which shows a flowchart of one aspect, in which a biological signal is recorded, and used to create a stimulus audio signal, which is sent to a speaker and instructs the user when to perform at least one repetitive motion activity. The biological signal (101) is amplified (102) and digitized (103). A processor (105) running a software algorithm (See FIG. 2) is responsible for creating an audio stimulus signal. It optionally stores the biological signal recording in memory (104) and also uses the memory to store settings and preferences for creation of the stimulus. Optionally, an audio file (106) may be retrieved and incorporated or modified to create the stimulus signal. The signal is passed through a D/A converter (107), amplified (108), and played through a speaker (109) for the user to stimulate when the one or more repetitive motion activity is to occur.

The software in the processor is responsible for receiving the biological signals, performing beat detection and creating the stimulus.

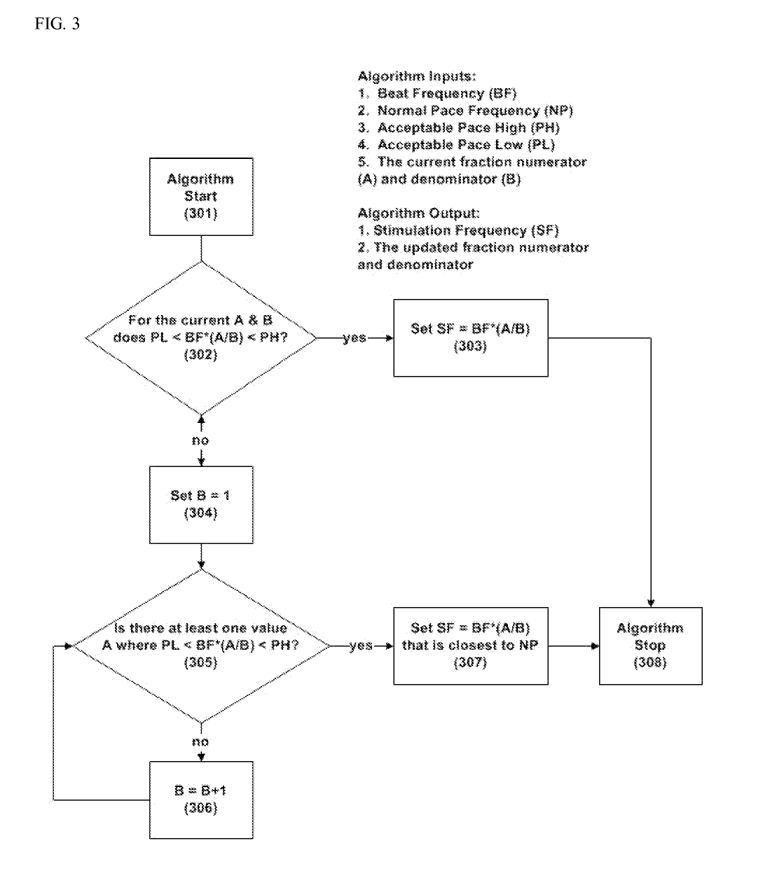

FIG. 2 shows a flowchart for the processor (105) from FIG. 1 in which a biological signal is processed. The intrinsic frequency of the biological signal is detected and filtered to remove transient variations. The stimulus algorithm from FIG. 3 is employed to create the timing of the stimulus signal. A phase component is added so that the stimulus is given at the optimal moment relative to the biological signal. The stimulus is created using an audio tone, music, or video, then combined and given as output to be presented to the user. In FIG. 2, a biological signal (201) is input to the processor. The software performs a beat detection (202) to find the precise timing of the biological signal. One non-limiting example would be determining heart beats using a peak detection algorithm on the incoming signal.

A smoothing filter (203) may be used to reduce the variability of the biological signals. One non-limiting example would be to remove the R-R beat variability of the detected heart beat. Smoothing makes it easier to calculate the stimulus frequency, and reduce its variability. Smoothing can be done by filtering the R-R values (time interval between beats) using a linear filter, and then creating the modified beat signal from the smoothed R-R values.

A stimulus algorithm (204) is used to create the stimulus beat (See FIG. 3). The stimulus beat may go through a phase buffering (205), which causes the actual stimulus to be delivered at the optimal point in the cycle of the biological signal. One non-limiting example would be delaying the foot-fall of a runner until a point in between heart beats, which helps to reduce blood pressure during the beat and reduces stress on the heart.

Stimulus creation (207) may be performed by making an audio or visual signal that incorporates the same beat as the stimulus beat signal. This uses a music, audio, or video file from memory (206). These may be combined (208) into a single stimulus signal (209) that can be sent to the output device relating to the stimulus method. Non-limiting examples include speakers, tactile stimulation, video monitors, LEDs, and LCDs.

The algorithm is responsible for accepting the filtered beat signal frequency and determining the optimal pace frequency for the repetitive motion activity. Every activity has a normal pace frequency, which is determined by the nature of the activity and the preference of the user. It is the pace the user would expect to maintain without any stimulation feedback. Two non-limiting examples would be running and freestyle swimming. The average pace of a competitive runner is usually approximately 180 steps per minute. In freestyle swimming, by comparison, the average stroke rate is closer to 60 strokes per minute. Normal pace frequency can also depend on at least one of height, weight, and ability of the athlete, as well as environmental influences, including, but not limited to, temperature, altitude, and slope. Therefore, in general the user should set the normal pace frequency for his/her activity.

The user will also have a maximum and minimum acceptable pace frequency for the activity. One non-limiting example would be running. A user may be comfortable with a pace between 160 and 200 steps per minute. Anything above 200, the high acceptable pace (PH), would require the user to chop his/her steps, and anything below 160, the low acceptable pace (PL), would require too long of a stride to be efficient.

The purpose of the algorithm is to find the stimulation frequency that is a common fraction of harmonics and sub-harmonics of the beat frequency and in the acceptable range between minimum and maximum pace frequency, in which the denominator is as small as possible. In one non-limiting example, if the normal pace frequency (NP) of a runner is 190 steps per minute (spm), and the acceptable range for the runner is between 150 and 200 spm, and his/her heart rate is 120 bpm, then the fraction with the lowest denominator when multiplied by the heart rate that falls within the acceptable range is 3/2. The stimulation frequency in this case is 120*(3/2)=180 spm.

FIG. 3 shows an example of a flowchart for an algorithm to create the stimulation frequency. In this, a fraction (A/B) is multiplied by the Beat Frequency (BF) of the biological system to create the stimulation frequency (SF). If the current fraction is adequate to keep the SF in the acceptable range for the user, the fraction does not change. However, if the current SF moves out of the acceptable range, a new fraction is chosen that has the lowest denominator possible, while allowing for a SF that falls in the acceptable range. The value that falls closest to the optimal pacing frequency is chosen as the new SF. At the start of the algorithm (301), certain parameters are received as inputs: Beat frequency (BF), NP, PH, PL, and the fraction that is currently being used to generate stimulation frequency (numerator A, and denominator B). The algorithm can be run at regular intervals to update the stimulation frequency as the biological frequency varies. Some non-limiting examples would be once per second, once every 5 seconds, and once per beat of the biological activity.

In the beginning, the algorithm checks to see if the updated stimulation frequency (SF) as calculated using the existing fraction (A/B) and the updated BF falls in the acceptable range (302). If so, then the SF is updated (303) and the algorithm ends (308). Otherwise, the algorithm goes through a loop, starting with B=1 (304), then checking to see if there is any value of A where BF*(A/B) falls in the acceptable range between PL and PH (305). If so, then the algorithm stops and finds the SF closest to NP (307). Otherwise, the algorithm increments B and checks again (306). This process is continued until values for A and B are found that result in an acceptable SF. At the end of the algorithm (308), the values for updated SF, A, and B are stored.

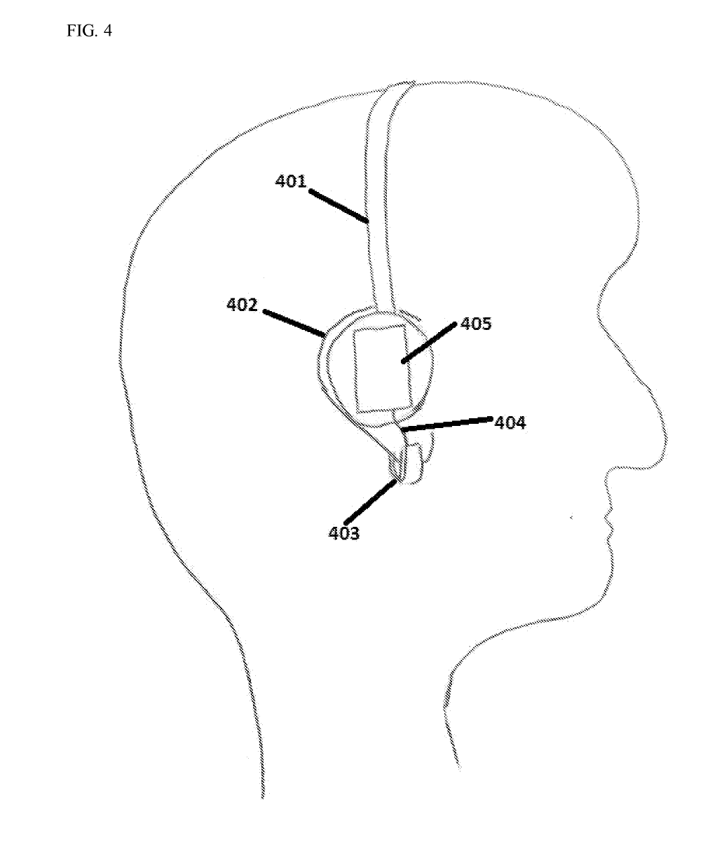

FIG. 4 shows one aspect, where an ear-clip pulse oximeter records the heart rate, and electronics and processor are embedded in or on one of the earphones. The processor creates the stimulation audio waveform, which is played through the headphones to the user. In FIG. 4 the heartbeat is detected using a pulse oximeter (403) clipped to the user's ear. The biological signal is sent via a wire (404) to the processor and supporting electronics (405), located on a set of headphones (401) that are placed on the user's ears (402). In this case, the stimulation signal would be an audio signal.

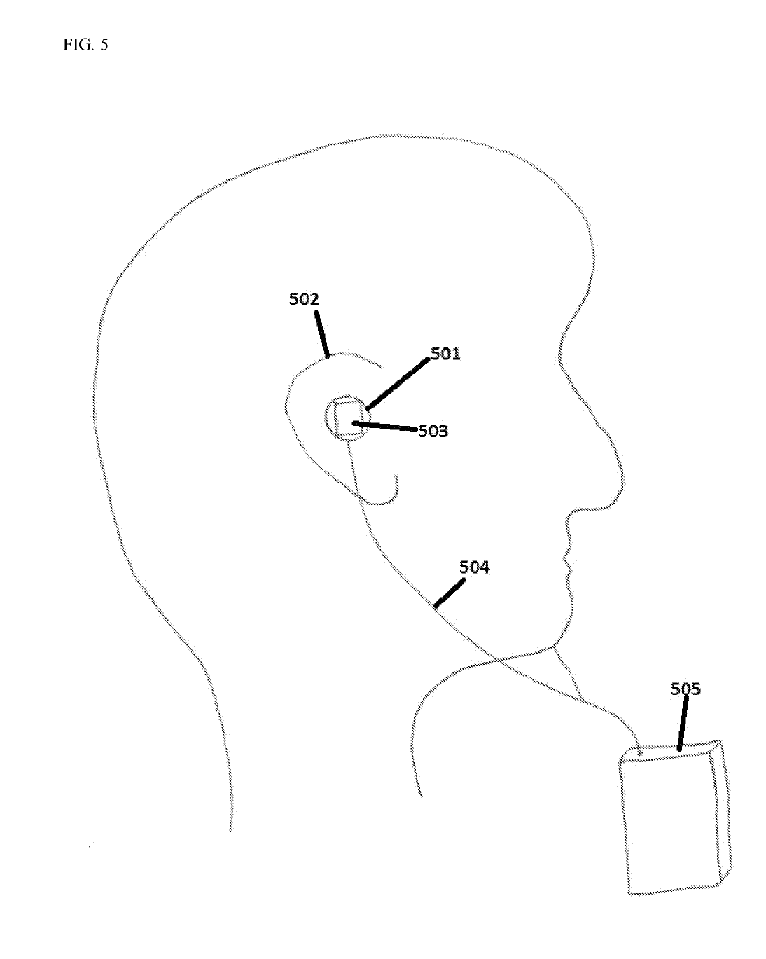

FIG. 5 shows an alternate aspect, in which a pulse oximeter is inserted into the ear with earphones. The beat signal is sent to a portable device such as a cellular phone or PDA, which has an embedded application that creates the stimulation signal, which is played back through the headphones. The pulse oximeter is inserted into the person's ear (502) on a set of earphones. Processing is done using a portable device such as a cellular phone. The pulse oximeter is on one or more of the headphones (501). An amplifier (503) conditions the biological signal before sending it through a wire (504) to the cellular phone (505). An application on the phone processes the signal and generates an audio stimulus signal that is sent back through a wire (504) and played into the user's ears (502). Optionally, a screen on the cellular phone can provide visual stimulus either in addition to or in substitution for the audio signal.

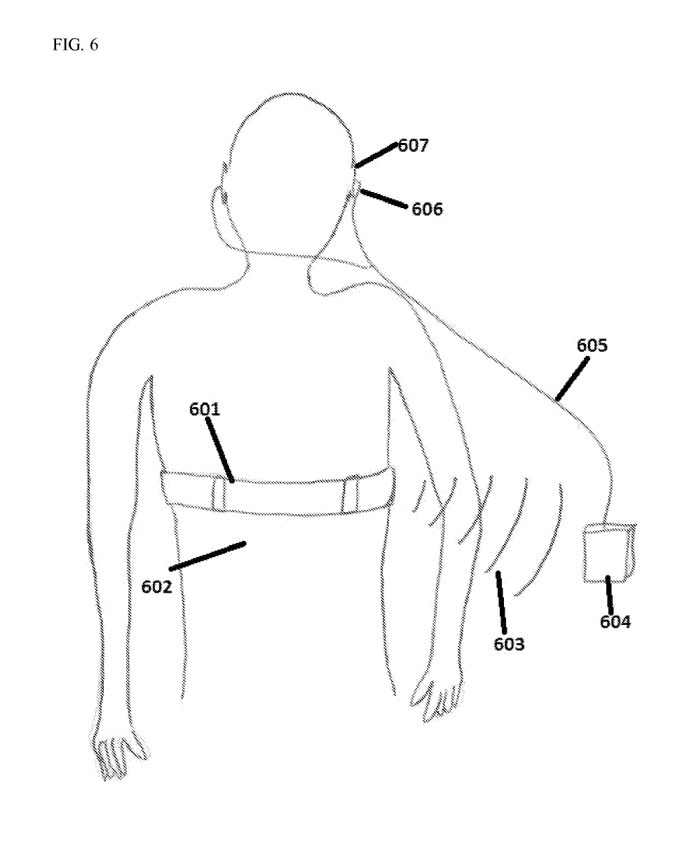

FIG. 6 shows an alternate aspect, in which a torso-mounted ECG sensor transmits the beat signal to the portable device such as a cellular phone, which has an embedded application that creates the stimulation signal, which is played back through headphones. The heartbeat is detected using a torso-mounted ECG strap (601) that is wrapped around the upper torso (602) of the user. The ECG strap communicates wirelessly (603) with a handheld device (604) to send beat information. An application on the handheld device processes the signal and generates an audio stimulus signal that is sent through a wire (605) and played through headphones (606) into the user's ears (607). Optionally, a screen on the handheld device can provide visual stimulus either in addition to or in substitution for the audio signal.

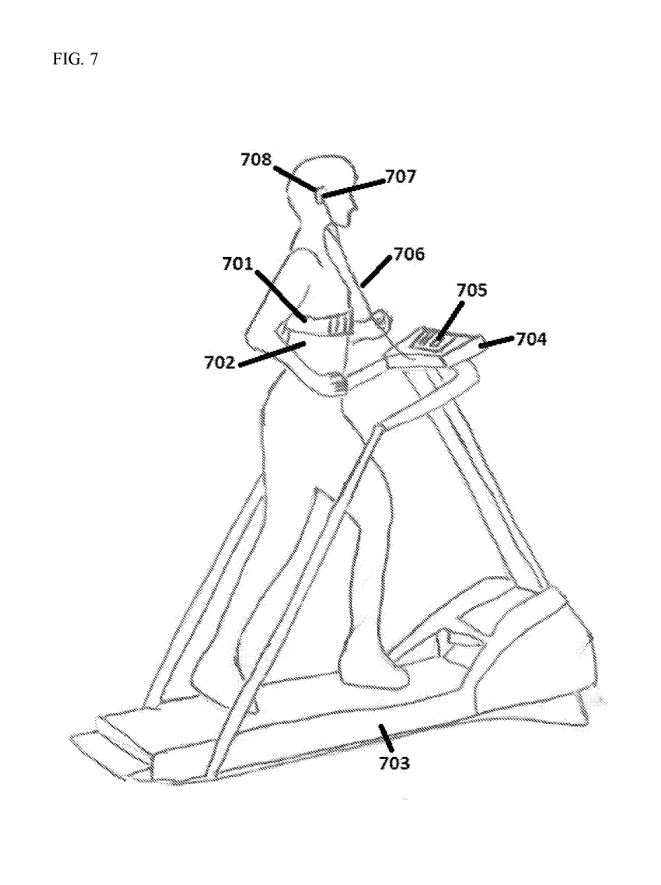

FIG. 7 shows an alternate aspect, in which the user is on a treadmill. A torso-mounted ECG sensor transmits the beat signal to a receiver in or on the treadmill control panel. There, a processor with embedded software creates the stimulation signal, which is played back through headphones. In addition, a flashing light is included on the control panel to provide visual stimulation to allow the user to time footfalls correctly. The user is on an exercise treadmill (703). The heartbeat is detected using a torso-mounted ECG strap (701) that is wrapped around the upper torso (702) of the user. The ECG strap communicates wirelessly with a receiver located in the console of the treadmill (704). Stimulus feedback is presented to the user through at least one of the display on the console (705) and through a wire (706) to headphones (707) in the ears of the user (708).

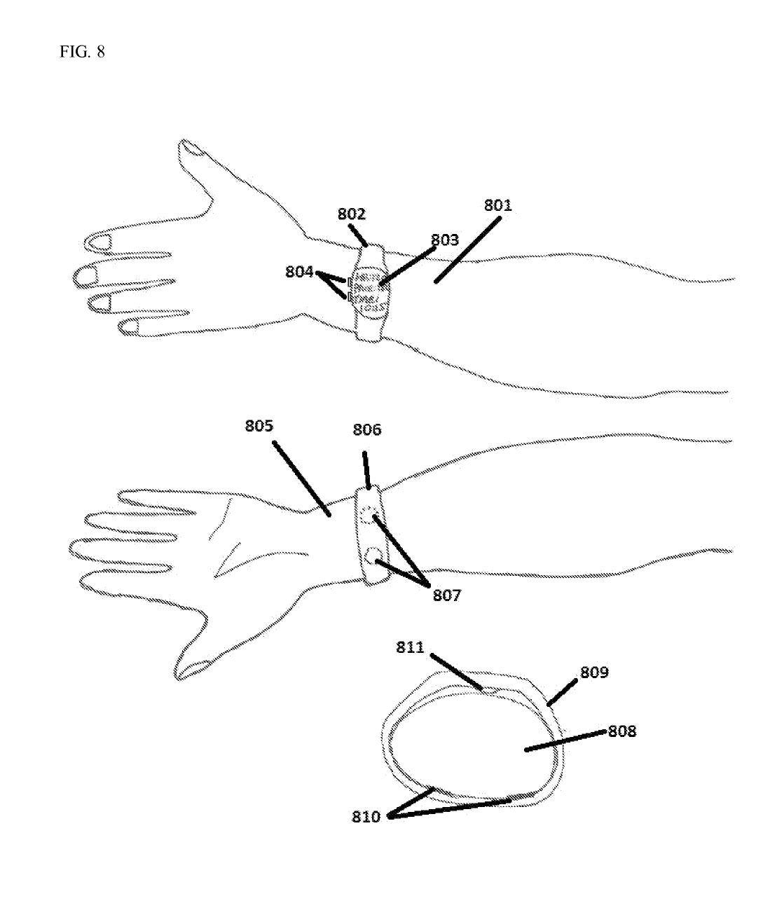

FIG. 8 shows an alternate aspect, in which the device is contained in a wristband. The heart rate is sensed through the wrist, either using ECG electrodes mounted in the wristband or with a pulse oximeter. The beat signal is sent to a processor in the face of the wristband, which allows setting of parameters and displays the heart rate, the stimulation frequency, and the elapsed time of the activity. The stimulation is performed using a tapping of the wrist through a thin diaphragm under the face of the device. The device is worn on the wrist and incorporated into a wristband and display. The strap (802) wraps around the wrist of the user with the display (803) on the back of the forearm (801). The display contains at least one of the current heart rate, the current pace stimulation frequency, and the elapsed time in the activity. All parameters can be set using pushbuttons on the display (804). The strap (806) underside of the wrist (805) contains at least one of electrodes and a pulse oximeter that senses the heartbeat of the user. A cross-sectional view of the wrist (808) shows the strap (809) with at least one of two electrodes and a pulse oximeter (810). In order to provide stimulation, a small tapping sensation is delivered through a diaphragm underneath the face of the device (811) delivered to the top of the wrist.

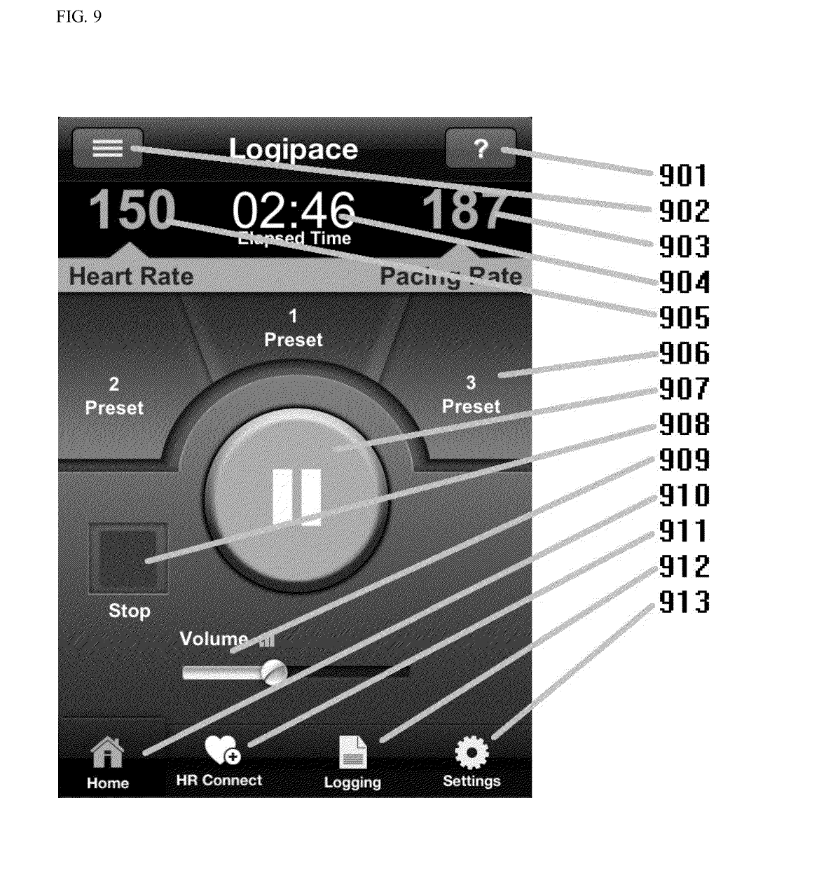

FIG. 9 shows an alternate aspect, in which the device is incorporated into a smart phone application, which senses heart rate and determines the pacing rate, allowing the user to start, pause, and stop the pacing stimulation via the touch screen. The device is implemented on a smartphone. The drawing shows the main screen (910) of the application that displays the recorded heart rate (905) and the pacing rate (903) as found using the algorithm from FIG. 3. The elapsed time of the activity (904) is displayed as well. The user is allowed set parameters such as PH, PL, and NP using a separate settings screen (913), and to retrieve logging information (902, 912). The user may store and retrieve parameters using one of three Presets (906). The user can start and pause the pacing stimulation (907) and stop the session (908). Volume of the pacing sound can also be adjusted (909). The user can connect to a wireless heart rate monitor (911). A help feature is available to describe the functions of the application (901).

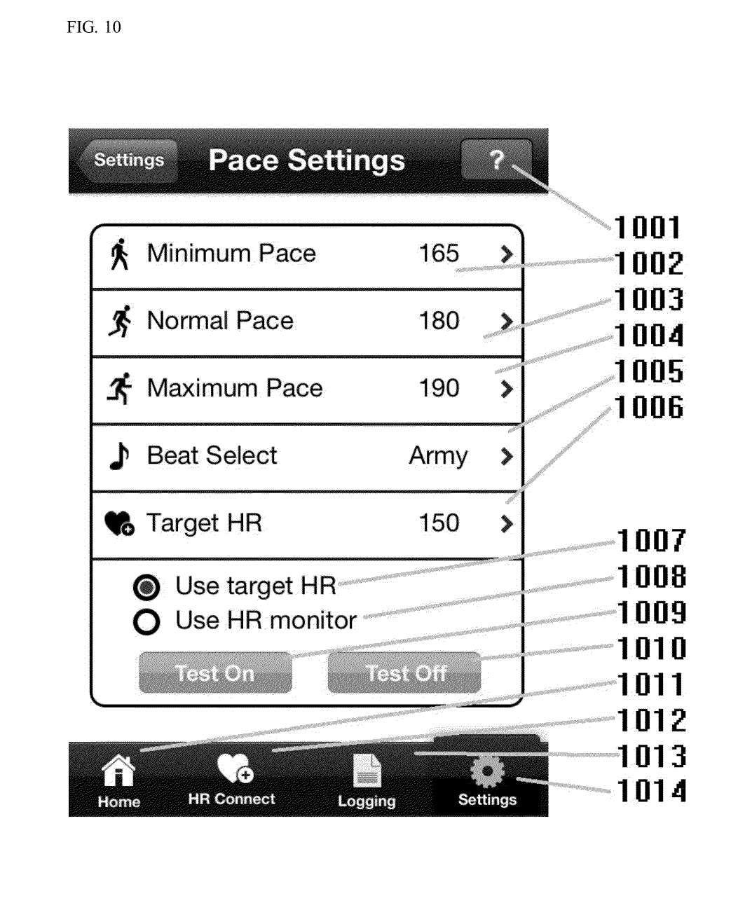

FIG. 10 shows an alternate aspect, in which the device is incorporated into a smart phone application, as in FIG. 9, where the user enters pace settings parameters, chooses whether the pacing frequency is based on a heart rate monitor input or a target heart rate setting, and is able to test the pacing sound at the normal pace frequency. The device is implemented on a smart phone as in FIG. 9, which allows the user to enter pacing parameters (1014). The user can enter the minimum pace PL (1002), the normal pace NP (1003), and the maximum pace PH (1004). The user can select from a variety of pacing sounds, such as army, hip-hop, techno, and rock (1005). The user has a choice of using a heart monitor to continuously update the pacing frequency during a run (1008), or to enter a target heart rate (1006) and use that to calculate a single pacing frequency that will stay the same throughout the run (1007). The user is able to test the pacing sound at the normal pace NP (1009) and to stop the test (1010). Note that the test feature allows the user to experiment with various settings to determine their specific PL, NP, and PH setting. The user may proceed back to the home screen as shown in FIG. 9 (1011), connect to a wireless heart rate monitor (1012), and to retrieve logging information (1013).

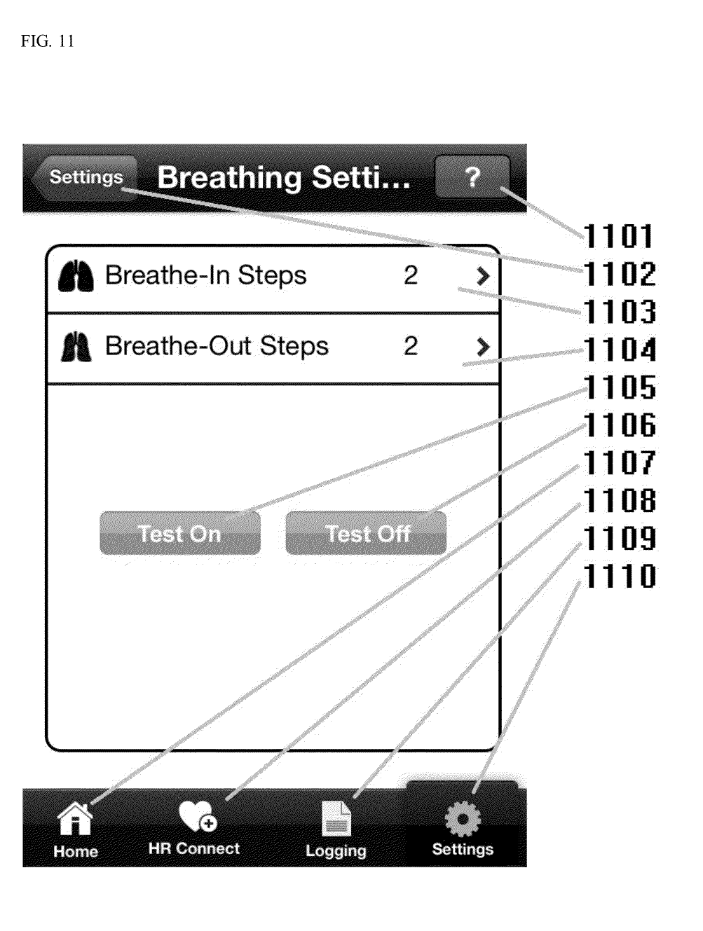

FIG. 11 shows an alternate aspect, in which the device is incorporated into a smart phone application, as in FIG. 9 and FIG. 10, where the user enters breathing settings parameters, indicating the number of steps to breathe in and the number of steps to breathe out, and to test the pacing sound with those parameters at the normal pace frequency. The device is implemented on a smart phone as in FIG. 9 and FIG. 10, which allows the user to enter breathing parameters (1102, 1110). The user enters the number of steps to breathe in (1103) and the number of steps to breathe out (1104). During the run, the device will play two distinct beats, one to indicate to the user when to breathe in and one to indicate when to breathe out. In this example, the user breathes in for two steps and breathes out for two steps. If, for example, the device uses a tick sound for breathe in and a tock sound for breathe out, then the device will deliver a tick-tick-tock-tock-tick-tick-tock-tock pacing rhythm and the specific pacing frequency. The user is able to test the pacing sound with the breathing settings at the normal pace NP (1105) and to stop the test (1106). The user may proceed back to the home screen as shown in FIG. 9 (1107), connect to a wireless heart rate monitor (1108), and to retrieve logging information (1109).

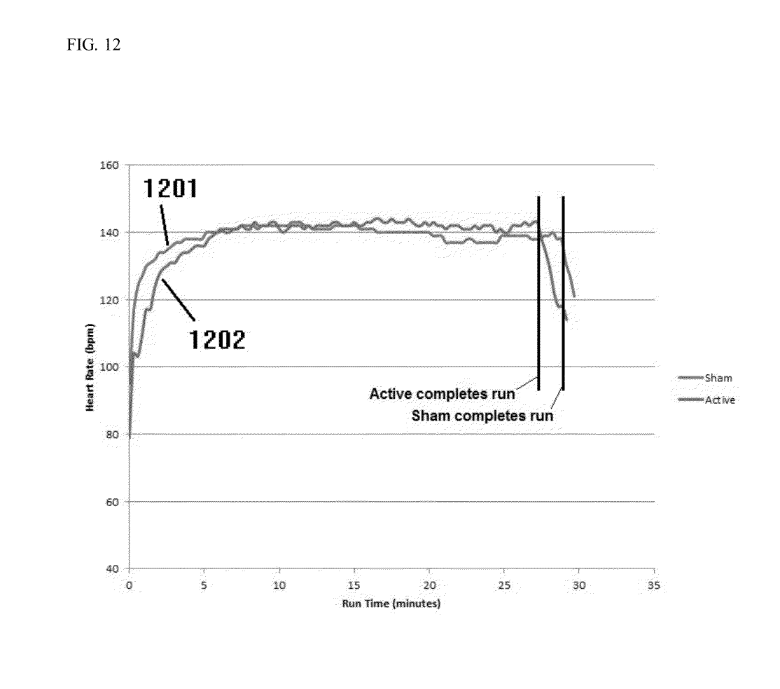

FIG. 12 shows a representative example of a subject's heart rate during sham (1201) versus active pacing (1202) during a clinical trial using pacing adapted to the subject's heart beat during running, as provided herein. The heart rate increases more slowly using active pacing, and the subject completes the run more quickly when using active pacing. The clinical trial investigated performance benefits from synchronizing a runner's strides to their heart rate using the algorithm shown in FIG. 3, referred to herein as adaptive paced cardiolocomotor synchronization (CLS). The algorithm was implemented on an iPhone 4, which generated a ‘tick-tock’ sounds through the iPhone's headphones. A sham-controlled crossover study was performed with 15 volunteers of various fitness levels. Subjects ran a 3 mile simulated training run at their normal pace on two consecutive days, randomized to one active pacing and one sham. Active pacing resulted in faster run times, lower heart rate variation, and a slower increase in heart rate, all of which indicate that running in sync with heart rate lessens the strain on the body as a whole, allowing the body to work less hard, and improving performance. The table below gives the results, showing cumulative run times in minutes (min) and seconds (s), heart rate (HR) variation coefficient, and the exponential time constant for the increase in heart rate for the active and sham condition. All values are expressed as mean (±SD) as applicable.

| | Sham | Active | P-value |
|---|---|---|---|
| 1st mile time (min:s) | 8:28 (1:08) | 8:29 (1:18) | 0.87 |
| 2nd mile time (min:s) | 17:41 (2:28) | 17:25 (2:34) | 0.08 |
| 3rd mile time (min:s) | 26:38 (3:31) | 26:03 (3:23) | 0.02 |
| HR variation coefficient | 0.03 | 0.01 | 0.09 |
| Time constant | 0.99 (0.30) | 1.53 (0.34) | 0.00 |

In general, subjects ran 3 miles in a faster time using active pacing compared with a self-selected pace. Run times did not reach statistical significance until the third mile, which would indicate a potential benefit for longer runs, where cardiac efficiency plays a greater role. Subjects generally reported that running was easier when using adaptive pacing. In every case, the heart rate rose more slowly during active pacing. The heart rate variability was generally lower while being paced, though not at a level to reach statistical significance, which may have had beneficial effects for sustained blood perfusion and oxygen uptake. See Phillips & Jin, Effect of Adaptive Paced Cardiolocomotor Synchronization during Running: A Preliminary Study, Journal of Sports Science and Medicine 11 (2013), In Press.

While the invention has been described in connection with certain preferred embodiments, other embodiments would be understood by one of ordinary skill in the art and are encompassed herein.

The methods and systems described herein may be deployed in part or in whole through a machine that executes computer software, program codes, and/or instructions on a processor. The present invention may be implemented as a method on the machine, as a system or apparatus as part of or in relation to the machine, or as a computer program product embodied in a computer readable medium executing on one or more of the machines. The processor may be part of a server, client, network infrastructure, mobile computing platform, stationary computing platform, or other computing platform. A processor may be any kind of computational or processing device capable of executing program instructions, codes, binary instructions and the like. The processor may be or include a signal processor, digital processor, embedded processor, microprocessor or any variant such as a co-processor (math co-processor, graphic co-processor, communication co-processor and the like) and the like that may directly or indirectly facilitate execution of program code or program instructions stored thereon. In addition, the processor may enable execution of multiple programs, threads, and codes. The threads may be executed simultaneously to enhance the performance of the processor and to facilitate simultaneous operations of the application. By way of implementation, methods, program codes, program instructions and the like described herein may be implemented in one or more thread. The thread may spawn other threads that may have assigned priorities associated with them; the processor may execute these threads based on priority or any other order based on instructions provided in the program code. The processor may include memory that stores methods, codes, instructions and programs as described herein and elsewhere. The processor may access a storage medium through an interface that may store methods, codes, and instructions as described herein and elsewhere. The storage medium associated with the processor for storing methods, programs, codes, program instructions or other type of instructions capable of being executed by the computing or processing device may include but may not be limited to one or more of a CD-ROM, DVD, memory, hard disk, flash drive, RAM, ROM, cache and the like.

A processor may include one or more cores that may enhance speed and performance of a multiprocessor. In embodiments, the process may be a dual core processor, quad core processors, other chip-level multiprocessor and the like that combine two or more independent cores (called a die).

The methods and systems described herein may be deployed in part or in whole through a machine that executes computer software on a server, client, firewall, gateway, hub, router, or other such computer and/or networking hardware. The software program may be associated with a server that may include a file server, print server, domain server, internet server, intranet server and other variants such as secondary server, host server, distributed server and the like. The server may include one or more of memories, processors, computer readable media, storage media, ports (physical and virtual), communication devices, and interfaces capable of accessing other servers, clients, machines, and devices through a wired or a wireless medium, and the like. The methods, programs or codes as described herein and elsewhere may be executed by the server. In addition, other devices required for execution of methods as described in this application may be considered as a part of the infrastructure associated with the server.

The server may provide an interface to other devices including, without limitation, clients, other servers, printers, database servers, print servers, file servers, communication servers, distributed servers and the like. Additionally, this coupling and/or connection may facilitate remote execution of program across the network. The networking of some or all of these devices may facilitate parallel processing of a program or method at one or more location without deviating from the scope of the invention. In addition, any of the devices attached to the server through an interface may include at least one storage medium capable of storing methods, programs, code and/or instructions. A central repository may provide program instructions to be executed on different devices. In this implementation, the remote repository may act as a storage medium for program code, instructions, and programs.

The software program may be associated with a client that may include a file client, print client, domain client, internet client, intranet client and other variants such as secondary client, host client, distributed client and the like. The client may include one or more of memories, processors, computer readable media, storage media, ports (physical and virtual), communication devices, and interfaces capable of accessing other clients, servers, machines, and devices through a wired or a wireless medium, and the like. The methods, programs or codes as described herein and elsewhere may be executed by the client. In addition, other devices required for execution of methods as described in this application may be considered as a part of the infrastructure associated with the client.

The client may provide an interface to other devices including, without limitation, servers, other clients, printers, database servers, print servers, file servers, communication servers, distributed servers and the like. Additionally, this coupling and/or connection may facilitate remote execution of program across the network. The networking of some or all of these devices may facilitate parallel processing of a program or method at one or more location without deviating from the scope of the invention. In addition, any of the devices attached to the client through an interface may include at least one storage medium capable of storing methods, programs, applications, code and/or instructions. A central repository may provide program instructions to be executed on different devices. In this implementation, the remote repository may act as a storage medium for program code, instructions, and programs.

The methods and systems described herein may be deployed in part or in whole through network infrastructures. The network infrastructure may include elements such as computing devices, servers, routers, hubs, firewalls, clients, personal computers, communication devices, routing devices and other active and passive devices, facilitys and/or components as known in the art. The computing and/or non-computing device(s) associated with the network infrastructure may include, apart from other components, a storage medium such as flash memory, buffer, stack, RAM, ROM and the like. The processes, methods, program codes, instructions described herein and elsewhere may be executed by one or more of the network infrastructural elements.

The methods, program codes, and instructions described herein and elsewhere may be implemented on a cellular network having multiple cells. The cellular network may either be frequency division multiple access (FDMA) network or code division multiple access (CDMA) network. The cellular network may include mobile devices, cell sites, base stations, repeaters, antennas, towers, and the like. The cell network may be a GSM, GPRS, 3G, EVDO, mesh, or other networks types.

The methods, programs codes, and instructions described herein and elsewhere may be implemented on or through mobile devices. The mobile devices may include navigation devices, cell phones, mobile phones, mobile personal digital assistants, laptops, palmtops, netbooks, pagers, electronic books readers, music players and the like. These devices may include, apart from other components, a storage medium such as a flash memory, buffer, RAM, ROM and one or more computing devices. The computing devices associated with mobile devices may be enabled to execute program codes, methods, and instructions stored thereon. Alternatively, the mobile devices may be configured to execute instructions in collaboration with other devices. The mobile devices may communicate with base stations interfaced with servers and configured to execute program codes. The mobile devices may communicate on a peer to peer network, mesh network, or other communications network. The program code may be stored on the storage medium associated with the server and executed by a computing device embedded within the server. The base station may include a computing device and a storage medium. The storage device may store program codes and instructions executed by the computing devices associated with the base station.

The computer software, program codes, and/or instructions may be stored and/or accessed on machine readable media that may include: computer components, devices, and recording media that retain digital data used for computing for some interval of time; semiconductor storage known as random access memory (RAM); mass storage typically for more permanent storage, such as optical discs, forms of magnetic storage like hard disks, tapes, drums, cards and other types; processor registers, cache memory, volatile memory, non-volatile memory, optical storage such as CD, DVD; removable media such as flash memory (e.g. USB sticks or keys), floppy disks, magnetic tape, paper tape, punch cards, standalone RAM disks, Zip drives, removable mass storage, off-line, and the like; other computer memory such as dynamic memory, static memory, read/write storage, mutable storage, read only, random access, sequential access, location addressable, file addressable, content addressable, network attached storage, storage area network, bar codes, magnetic ink, and the like.

The methods and systems described herein may transform physical and/or or intangible items from one state to another. The methods and systems described herein may also transform data representing physical and/or intangible items from one state to another.

The elements described and depicted herein, including in flow charts and block diagrams throughout the figures, imply logical boundaries between the elements. However, according to software or hardware engineering practices, the depicted elements and the functions thereof may be implemented on machines through computer executable media having a processor capable of executing program instructions stored thereon as a monolithic software structure, as standalone software facilitys, or as facilitys that employ external routines, code, services, and so forth, or any combination of these, and all such implementations may be within the scope of the present disclosure. Examples of such machines may include, but may not be limited to, personal digital assistants, laptops, personal computers, mobile phones, other handheld computing devices, medical equipment, wired or wireless communication devices, transducers, chips, calculators, satellites, tablet PCs, electronic books, gadgets, electronic devices, devices having artificial intelligence, computing devices, networking equipments, servers, routers and the like. Furthermore, the elements depicted in the flow chart and block diagrams or any other logical component may be implemented on a machine capable of executing program instructions. Thus, while the foregoing drawings and descriptions set forth functional aspects of the disclosed systems, no particular arrangement of software for implementing these functional aspects should be inferred from these descriptions unless explicitly stated or otherwise clear from the context. Similarly, it will be appreciated that the various steps identified and described above may be varied, and that the order of steps may be adapted to particular applications of the techniques disclosed herein. All such variations and modifications are intended to fall within the scope of this disclosure. As such, the depiction and/or description of an order for various steps should not be understood to require a particular order of execution for those steps, unless required by a particular application, or explicitly stated or otherwise clear from the context.

The methods and/or processes described above, and steps thereof, may be realized in hardware, software or any combination of hardware and software suitable for a particular application. The hardware may include a general purpose computer and/or dedicated computing device or specific computing device or particular aspect or component of a specific computing device. The processes may be realized in one or more microprocessors, microcontrollers, embedded microcontrollers, programmable digital signal processors or other programmable device, along with internal and/or external memory. The processes may also, or instead, be embodied in an application specific integrated circuit, a programmable gate array, programmable array logic, or any other device or combination of devices that may be configured to process electronic signals. It will further be appreciated that one or more of the processes may be realized as a computer executable code capable of being executed on a machine readable medium.

The computer executable code may be created using a structured programming language such as C, an object oriented programming language such as C++, or any other high-level or low-level programming language (including assembly languages, hardware description languages, and database programming languages and technologies) that may be stored, compiled or interpreted to run on one of the above devices, as well as heterogeneous combinations of processors, processor architectures, or combinations of different hardware and software, or any other machine capable of executing program instructions.

Thus, in one aspect, each method described above and combinations thereof may be embodied in computer executable code that, when executing on one or more computing devices, performs the steps thereof. In another aspect, the methods may be embodied in systems that perform the steps thereof, and may be distributed across devices in a number of ways, or all of the functionality may be integrated into a dedicated, standalone device or other hardware. In another aspect, the means for performing the steps associated with the processes described above may include any of the hardware and/or software described above. All such permutations and combinations are intended to fall within the scope of the present disclosure.

While the invention has been disclosed in connection with the preferred embodiments shown and described in detail, various modifications and improvements thereon will become readily apparent to those skilled in the art. Accordingly, the spirit and scope of the present invention is not to be limited by the foregoing examples, but is to be understood in the broadest sense allowable by law.

The various functions or processes disclosed herein (such as, for non-limiting example, logic that performs a function or process) may be described as data and/or instructions embodied in various computer-readable media, in terms of their behavioral, register transfer, logic component, transistor, layout geometries, and/or other characteristics. The logic described herein may comprise, according to various embodiments of the invention, software, hardware, or a combination of software and hardware. The logic described herein may comprise computer-readable media, Computer-readable media in which such formatted data and/or instructions may be embodied include, but are not limited to, non-volatile storage media in various forms (e.g., optical, magnetic or semiconductor storage media) and carrier waves that may be used to transfer such formatted data and/or instructions through wireless, optical, or wired signaling media or any combination thereof. Examples of transfers of such formatted data and/or instructions by carrier waves include, but are not limited to, transfers (uploads, downloads, e-mail, etc.) over the Internet and/or other computer networks via one or more data transfer protocols (e.g., HTTP, FTP, SMTP, etc.). When received within a computer system via one or more computer-readable media, such data and/or instruction-based expressions of components and/or processes under the ICS may be processed by a processing entity (e.g., one or more processors) within the computer system in conjunction with execution of one or more other computer programs.

Unless the context clearly requires otherwise, throughout the description and the claims, the words “comprise,” “comprising,” and the like are to be construed in an inclusive sense as opposed to an exclusive or exhaustive sense; that is to say, in a sense of “including, but not limited to.” Words using the singular or plural number also include the plural or singular number respectively. Additionally, the words “herein,” ‘hereunder,” “above,” “below,” and words of similar import refer to this application as a whole and not to any particular portions of this application. When the word “or” is used in reference to a list of two or more items, that word covers all of the following interpretations of the word: any of the items in the list, all of the items in the list and any combination of the items in the list.

The above descriptions of illustrated embodiments of the system, methods, or devices are not intended to be exhaustive or to be limited to the precise form disclosed. While specific embodiments of, and examples for, the system, methods, or devices are described herein for illustrative purposes, various equivalent modifications are possible within the scope of the system, methods, or devices, as those skilled in the relevant art will recognize. The teachings of the system, methods, or devices provided herein can be applied to other processing systems, methods, or devices, not only for the systems, methods, or devices described.

The elements and acts of the various embodiments described can be combined to provide further embodiments. These and other changes can be made to the system in light of the above detailed description.

In general, in the following claims, the terms used should not be construed to limit the system, methods, or devices to the specific embodiments disclosed in the specification and the claims, but should be construed to include all processing systems that operate under the claims. Accordingly, the system, methods, and devices are not limited by the disclosure, but instead the scopes of the system, methods, or devices are to be determined entirely by the claims.

While certain aspects of the system, methods, or devices are presented below in certain claim forms, the inventors contemplate the various aspects of the system, methods, or devices in any number of claim forms. For example, while only one aspect of the system, methods, or devices is recited as embodied in machine-readable medium, other aspects may likewise be embodied in machine-readable medium. Accordingly, the inventors reserve the right to add additional claims after filing the application to pursue such additional claim forms for other aspects of the system, methods, or devices.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

All documents referenced herein are hereby incorporated by reference.

What is claimed is:

1. A method, comprising:
Determining at least one rhythmic biological signal;
Generating a rhythmic stimulus having a defined relationship to the biological signal; and
Presenting the rhythmic stimulus to a user to enable the user to perform a repetitive motion activity in response to the stimulus

2. A method of claim 1, wherein the frequency of the stimulus for the repetitive motion is a common fraction multiplied by the biological signal frequency.

3. A method of claim 1, wherein the frequency of the stimulus is near a common fraction multiplied by the biological signal frequency.

4. A method of claim 1, wherein the frequency of the stimulus for the repetitive motion is an integer multiple of the biological signal frequency.

5. A method of claim 1, wherein the use of the feedback stimulation allows the user to improve the performance of the activity.

6. A method of claim 1, wherein the use of the feedback stimulation allows the user to improve at least one of wellness, focus, and concentration.

7. A method of claim 1, wherein the use of feedback is adapted to reduce injuries.

8. A method of claim 1, wherein the use of the feedback stimulation causes at least one biological signal to become more regular and have fewer spurious or arrhythmic beats.

9. A method of claim 1, wherein the use of the feedback stimulation achieves results similar to meditation, allowing the user to develop a sense of calm and well-being, and to be less acutely aware of at least one of discomfort, fatigue, and muscle soreness as a result of the repetitive motion activity.

10. A method of claim 1, wherein the use of the feedback stimulation results in a reduction of symptoms of at least one of depression, anxiety, obsessive-compulsive, seizure, Parkinson's disease, ADHD, autism, substance abuse, head injury, Alzheimer's disease, eating disorder, sleep disorder, and tinnitus.

11. A method of claim 1, wherein a phase offset is added to the stimulation signal to adjust the timing of the repetitive motion activity relative to the phase of the rhythmic biological activity.

12. A method of claim 11, wherein the use of the phase offset results in lowered stress to the biological system.

13. A method of claim 1, wherein the feedback stimulation frequency is equal to a common fraction multiplied by the biological signal frequency, or a harmonic or sub-harmonic thereof.

14. A method of claim 13, wherein the feedback is at least one of audio, visual, and tactile stimulation.

15. A method of claim 14, wherein the feedback is audio feedback.

16. A method of claim 15, wherein audio feedback is at least one of beeps, tones, drum-beats, thumps, hisses, and voices.

17. A method of claim 15, wherein the audio feedback is presented as part of music, where the tempo of the music matches or is close to the desired feedback stimulation frequency.

18. A method of claim 15, wherein the tempo of existing music is modulated to match or be close to the desired feedback stimulation frequency.

19. A method of claim 15, wherein music with a tempo that matches or is close to the desired feedback stimulation frequency is selected from a set of music with various tempos.

20. A method of claim 15, wherein music with a tempo that matches or is within a specified range of the desired feedback stimulation frequency is selected from a set of music with various tempos, and then modulated to match or be close to the desired feedback stimulation frequency.

21. A method of claim 14, wherein the feedback is visual feedback.

22. A method of claim 21, wherein the visual feedback is at least one of flashing LED, flashing LCD, flashing icons, and movement of an object on a video screen.

23. A method of claim 22, wherein the visual feedback is presented with video entertainment, with the stimulation as part of the video.

24. A method of claim 14, wherein the feedback is tactile feedback.

25. A method of claim 24, wherein the tactile feedback is at least one of vibration and tapping.

26. A method of claim 24, wherein two or more feedback stimulations are given simultaneously, each corresponding to either the same biological signal or two or more different biological signals.

27. A method of claim 1, wherein the repetitive motion activity is at least one of breathing, running, bicycling, swimming, walking, hiking, marching, jumping rope, aerobics, dancing, boxing, rowing, hammering, and typing.

28. A method of claim 1, wherein the biological signal is at least one of heartbeat, breathing, and brainwaves.

29. A method of claim 28, wherein the heartbeat is recorded using at least one of electrocardiogram, pulse oximetry, and a microphone.

30. A method of claim 28, wherein the breathing is recorded using at least one of electromyography, torso girth sensor, microphone, and oxygen sensor.

31. A method of claim 28, wherein the brainwaves are recorded using at least one of an electroencephalograph (EEG) and a magneto encephalograph (MEG).

32. A method of claim 1, wherein the stimulation signal frequency is calculated as equal to, or a harmonic or sub-harmonic thereof, the biological signal frequency, multiplied by a common fraction with the lowest denominator, that falls within a pre-defined acceptable boundary and lies closest to a normal pace for the repetitive motion activity, where the normal pace is defined as the pace at which the activity would occur if no stimulation feedback were presented to the user.

33. A method of claim 32, wherein at least one of the type of stimulus, the normal pace frequency, the acceptable high pace frequency, and the acceptable low pace frequency for the repetitive motion activity may be set or adjusted by the user before, during, or after the activity.

34. A method of claim 32, wherein at least one of the type of stimulus, the normal pace frequency, the acceptable high pace frequency, and the acceptable low pace frequency for the repetitive motion activity may be set or adjusted automatically before or during the activity, based on at least one of the physical requirements of the activity and the physical state of the user.

35. A method of claim 34, wherein the variability in physical requirements of the repetitive motion activity include at least one of atmospheric temperature, altitude, slope, atmospheric humidity, the user's location, distance traveled, the user's speed, and the time duration of the activity.

36. A method of claim 35, wherein the physical state of the user include at least one of body temperature, heart rate, breathing rate, breathing volume, the user's brainwave activity, and the accuracy of matching the pacing stimulus.

37. A device, which records one or more biological signals and provides a rhythmic stimulus, which is presented to the user as feedback to allow the user to perform a repetitive motion activity such that the frequency of the repetitive motion matches or is close to a common fraction of the biological signal frequency, or a harmonic or sub-harmonic thereof.

38. A device of claim 37, wherein the biological signal is at least one of heartbeat, breathing, and brainwaves.

39. A device of claim 38, wherein the heart beat is recorded using at least one of ECG electrodes, a pulse oximeter, and a microphone.

40. A device of claim 38, wherein the breathing is recorded using at least one of electromyography, a torso girth sensor, oxygen sensor, and a microphone.

41. A device of claim 38, wherein the brainwaves are detected using at least one of an EEG and an MEG.

42. A device of claim 38, wherein the biological signal is transmitted either wired or wirelessly to a remote device that creates the rhythmic stimulus.

43. A device of claim 42, wherein the remote device is at least one of a cellular phone, a tablet PC, a personal digital assistant (PDA), a laptop PC, a desktop PC, a wristband, and electronics contained in exercise equipment.

44. A device of claim 37, wherein the stimulation feedback is presented to the user using at least one of an audio signal, a video signal, and a tactile sensation.

45. A device of claim 44, wherein the audio signal is played using at least one of headphones, earphones, and external speakers.

46. A device of claim 44, wherein audio feedback is at least one of beeps, tones, drum-beats, thumps, hisses, and voices.

47. A device of claim 44, wherein the audio feedback is presented as part of music, where the tempo of the music matches or is close to the desired feedback stimulation frequency.

48. A device of claim 44, wherein the video signal is presented using at least one of a flashing LED, flashing LCD, flashing icons, and movement of an object on a video screen.

49. A device of claim 48, wherein the visual feedback is presented with video entertainment, with the stimulation as part of the video.

50. A device of claim 44, wherein the tactile sensation is given by at least one of vibration and tapping.

51. A device of claim 37, wherein the device incorporates a means wherein change in altitude is detected and translated into a slope, and this information is used to optionally adjust at least one of type of stimulus, normal pace frequency, maximum acceptable pace frequency, and minimum acceptable pace frequency.

52. A device of claim 37, wherein the device incorporates a sensor to measure at least one of atmospheric temperature, body temperature, humidity, and wind, and this information is used to optionally adjust at least one of type of stimulus, normal pace frequency, maximum acceptable pace frequency, and minimum acceptable pace frequency.

53. A device of claim 37, wherein the device incorporates a means of sensing position, such as with a GPS or with another such mechanism and this information is used to optionally adjust at least one of type of stimulus, normal pace frequency, maximum acceptable pace frequency, and minimum acceptable pace frequency.

54. A device of claim 37, wherein the device announces to the user, either through audio or visual means, or both, at least one of average heart rate, peak heart rate, minimum heart rate, instantaneous heart rate, distance covered, calories burned, average distance per pace, average speed, average pace frequency, stimulus frequency, accuracy of pace to the specified pace, and motivational messages.
